(19) United States
(12) Patent Application Publication
(10) Pub. No.: US 2014/0276714 A1
(43) Pub. Date: Sep. 18, 2014

(54) ACTIVE INFUSION SHEATH FOR ULTRASOUND ABLATION CATHETER

(71) Applicant: BOSTON SCIENTIFIC SCIMED, INC., MAPLE GROVE, MN (US)

(72) Inventors: KEVIN D. EDMUNDS, HAM LAKE, MN (US); MICHAEL J. PIKUS, GOLDEN VALLEY, MN (US); MARK L. JENSON, GREENFIELD, MN (US)

(73) Assignee: BOSTON SCIENTIFIC SCIMED, INC., MAPLE GROVE, MN (US)

(21) Appl. No.: 14/194,347

(22) Filed: Feb. 28, 2014

Related U.S. Application Data
(60) Provisional application No. 61/787,714, filed on Mar. 15, 2013.

(51) Int. Cl. A61N 7/02

(52) U.S. Cl. CPC A61N 7/02
USPC 606/28

(57) ABSTRACT

Systems for nerve and tissue modulation are disclosed. An example system may include an intravascular nerve modulation system including an elongated shaft having a first tubular member and a second tubular member. Each of the tubular members may have a proximal end a distal end. The distal end of the second tubular member may be extended distally beyond the distal end of the first tubular member. The system may further include at least one transducer affixed to the distal end region of the second tubular member. In addition, the system may include an infusion sheath having a proximal end and a distal end and the proximal end of the infusion sheath may be fixedly secured to the catheter shaft adjacent the distal end of the first tubular member.

### CROSS-REFERENCE TO RELATED APPLICATIONS

This application claims priority under 35 U.S.C. §119 to U.S. Provisional Application Ser. No. 61/787,714, filed Mar. 15, 2013, the entirety of which is incorporated herein by reference.

### TECHNICAL FIELD

The present disclosure relates generally to methods and apparatuses for nerve modulation techniques such as ablation of nerve tissue or other modulation techniques through the walls of blood vessels.

### BACKGROUND

Certain treatments may require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using ultrasonic energy. In other instances, the perivascular nerves may be ablated by other means including application of thermal, radiofrequency, laser, microwave, and other related energy sources to the target region. Ultrasound transducers may dissipate some energy as heat into the blood and surrounding tissue as well as causing the ultrasound transducers to become hot. This may result in blood damage, clotting, and/or protein fouling of the transducer among other undesirable side effects. In some instances, overheating of the ultrasound transducer may result in the failure of the transducers. It may be desirable to provide for alternative systems and methods for intravascular nerve modulation with increased cooling of the transducers.

### SUMMARY

The present disclosure is directed to an intravascular nerve modulation system for performing nerve ablation.

Accordingly, one illustrative embodiment includes an intravascular nerve modulation system having a catheter shaft. The catheter shaft may include a first tubular member defining an infusion lumen and a second tubular member. Each of the tubular members may have a proximal end and a distal end. The distal end of the second tubular member may extend distally beyond the distal end of the first tubular member. The system may also include at least one ablation transducer affixed to the distal end region of the second tubular member. The ablation transducer may be cylindrical. The system may further include an infusion sheath having a proximal end and a distal end and may be configured such that the distal end may remain open. The proximal end of the infusion sheath may be fixedly secured to the catheter shaft adjacent the distal end of the first tubular member and may be configured to be disposed over the ablation transducer. The infusion sheath may include a sonically translucent material. The infusion sheath may further include one or more reinforcing filaments extending along its length.

Another illustrative embodiment includes an intravascular nerve modulation system that may include an outer tubular member and an inner tubular member, each having a proximal end and a distal end and a lumen extending therebetween. The inner tubular member may be disposed within the lumen of the outer tubular member. The distal end of the inner tubular member may extend distally beyond the distal end of the outer tubular member. Further, the system may include at least one ablation transducer affixed to the distal end region of the inner tubular member. The ablation transducer may have a cylindrical shape in one configuration. Furthermore, the intravascular modulation system may include an infusion sheath having a proximal end and a distal end. The proximal end of the infusion sheath may be fixedly secured adjacent to the distal end of the outer tubular member. The infusion sheath may be disposed over the ablation transducer. The infusion sheath may comprise a sonically translucent material and may include one or more reinforcing filaments extending along a length thereof.

In yet another illustrative embodiment, the intravascular nerve modulation system may include a first tubular member and a second tubular member, each having a proximal end, a distal end and a lumen extending therebetween. The second tubular member may extend longitudinally along the first tubular member such that the distal end of the second tubular member may extend distally beyond the distal end of the first tubular member. Further, the system may include at least one ablation transducer, affixed to the distal end region of the second tubular member. In addition, the intravascular modulation system may include an infusion sheath having a proximal end and a distal end. The proximal end of the infusion sheath may be secured to the system adjacent the distal end of the first tubular member. The lumen of the outer tubular member may be configured to transport an infusion fluid from the proximal end of the outer tubular member to the distal end of the outer tubular member and into the infusion sheath. The infusion sheath may comprise a sonically translucent material and may include one or more reinforcing filaments extending along a length thereof.

Although discussed with specific reference to use with the renal nerves of a patient, the intravascular nerve modulation systems in accordance with the disclosure may be adapted and configured for use in other parts of the anatomy, such as the nervous system, the circulatory system, or other parts of the anatomy of a patient.

The above summary of an example embodiment is not intended to describe each disclosed embodiment or every implementation of the present disclosure.

### BRIEF DESCRIPTION OF THE DRAWINGS

The disclosure may be more completely understood in consideration of the following detailed description of various embodiments in connection with the accompanying drawings, in which:

FIG. 1 illustrates an example renal nerve modulation system in situ.

FIG. 2 illustrates a side view of a portion of an example intravascular nerve modulation system disposed within a body lumen.

FIG. 3 illustrates a cross-section of the illustrative intravascular nerve modulation system of FIG. 2 disposed within a body lumen.

FIG. 4 illustrates a side view of a portion of another example intravascular nerve modulation system disposed within a body lumen.

FIG. 5 illustrates a side view of a portion of another example intravascular nerve modulation system disposed within a body lumen.

FIG. 6 illustrates a side view of a portion of another example of an intravascular nerve modulation system disposed within a body lumen.

FIG. 7 illustrates a cross-section of a portion of another example intravascular nerve modulation system.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may be indicative as including numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

Although some suitable dimensions ranges and/or values pertaining to various components, features and/or specifications are disclosed, one of the skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end further from the device operator during use.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless cleared stated to the contrary.

Certain treatments require the temporary or permanent interruption or modification of select nerve function. One example treatment is renal nerve ablation, which is sometimes used to treat conditions related to hypertension or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

While the systems and methods described herein are discussed relative to renal nerve modulation, it is contemplated that the systems and methods may be used in other locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with ultrasound ablation. The term modulation refers to ablation and other techniques that may alter the function of affected nerves.

Ultrasound energy may be used to generate heat at a target location. The high frequency acoustic waves produced by an ultrasonic transducer may be directed at a target region and absorbed at the target region. As the energy emitted is absorbed, temperature of the target region may rise. In order to perform renal nerve ablation, target nerves must be heated sufficiently to make them nonfunctional, while thermal injury to the artery wall is undesirable. Heating of the artery wall during the procedure may increase pain, which is also undesirable. When a portion of tissue is ablated, tissue properties change, and increased attenuation of the ultrasound energy can make ablation past this ablated tissue difficult. Ultrasound ablation catheters may also generate significant heat in the ultrasound transducer. That heat may consequently form blood clots on or around the transducer, damage the surrounding blood, and/or damage the transducers, among other undesirable side effects. As the ablation transducers heat, the energy conversion efficiency of those devices is lowered, thus generating even more heat. Thus, normal operations of ablation transducers may be characterized by increasingly lower efficiency during operation. The efficiency of the ablation transducers may be enhanced using a cooling mechanism. One possible cooling mechanism is passing an infusion fluid over the transducers.

FIG. 1 is a schematic view of an illustrative renal nerve modulation system 10 in situ. The renal nerve modulation system 10 may include an element 12 for providing power to a transducer disposed adjacent to, about, and/or within a central elongated shaft 14 and, optionally, within a guide catheter 16. A proximal end of the element 12 may be connected to a control and power element 18, which supplies the necessary electrical energy to activate the one or more transducers at or near a distal end of the element 12. The control and power element 18 may include monitoring elements to monitor parameters such as power, temperature, voltage, pulse size and/or frequency and other suitable parameters as well as suitable controls for performing the desired procedure. In some instances, the power element 18 may control an ultrasound ablation transducer. The ablation transducer may be configured to operate at a frequency of about 9-10 megahertz (MHz). It is contemplated that any desired frequency may be used, for example, from 1-20 MHz. In addition, it is contemplated that frequencies outside this range may also be used, as desired. While the term “ultrasound” is used herein, this is not meant to limit the range of vibration frequencies contemplated. For example, it is contemplated that the perivascular nerves may be ablated by other means including application of thermal, radiofrequency, laser, microwave, and other related energy sources, or combinations thereof to the target region. For example, the devices and methods described herein may be applied to devices utilizing frequencies outside of the ultrasound frequency range.

FIG. 2 is a side view and FIG. 3 is a cross-sectional view of an illustrative embodiment of a distal end portion of an intravascular nerve modulation system 100 disposed within a body lumen 102 having a vessel wall 104. Local body tissue (not shown) may surround the vessel wall 104. The local body tissue may comprise adventitia and connective tissues, nerves, fat, fluid, etc., in addition to the muscular vessel wall 104. A portion of the surrounding tissue may constitute the desired treatment region.

The system 100 may include an elongate shaft 106 having a distal end region 108. The elongate shaft 106 may extend proximally from the distal end region 108 to a proximal end configured to remain outside of a patient's body. The proximal end of the elongate shaft 106 may include a hub attached thereto for connecting other treatment devices or providing a port for facilitating other treatments. It is contemplated that the stiffness of the elongate shaft 106 may be modified to form a modulation system 100 for use in various vessel diameters and various locations within the vascular tree. The elongate shaft 106 may further include one or more lumens extending therethrough. For example, the elongate shaft 106 may include a guidewire lumen and/or one or more auxiliary lumens. In some instances, the elongate shaft 106 may include an infusion lumen, as will be discussed in more detail below. The lumens may be configured in any way known in the art. For example, the guidewire lumen may extend the entire length of the elongate shaft 106 such as in an over-the-wire catheter or may extend only along a distal portion of the elongate shaft 106 such as in a single operator exchange (SOE) catheter. These examples are not intended to be limiting, but rather examples of some possible configurations. While not explicitly shown, the modulation system 100 may further include temperature sensors/wire, an infusion lumen, radiopaque marker bands, fixed guidewire tip, a guidewire lumen, external sheath, centering basket, and/or other components to facilitate the use and advancement of the system 100 within the vasculature.

In some embodiments, the elongated catheter shaft 106 may have a relatively long, thin, flexible tubular configuration. In some instances, the elongated shaft 106 may have a generally circular cross-section, however, other suitable configurations such as, but not limited to, rectangular, oval, irregular, or the like may also be contemplated. In addition, the elongated shaft 106 may have a cross-sectional configuration adapted to be received in a desired vessel, such as a renal artery. For instance, the elongated shaft 106 may be sized and configured to accommodate passage through the intravascular path, which leads from a percutaneous access site in, for example, the femoral, brachial, or radial artery, to a targeted treatment site, for example, within a renal artery.

The elongated shaft 106 may include a first tubular member 110 and a second tubular member 112. The first tubular member 110 may have a proximal end (not shown), a distal end 114, a distal end region 116 and a lumen 118 (as shown in FIG. 3) extending between the proximal end and the distal end. In some embodiments, the lumen 118 may be an infusion lumen and may be in fluid communication with an infusion fluid source configured to remain outside of a patient's body. The second tubular member 112 may have a proximal end (not shown), a distal end 122, and a lumen 124 extending therebetween. In some embodiments, the lumen 124 of the second tubular member may be a guidewire lumen. The distal end region 126 of the second tubular member 112 may extend distally beyond the distal end 114 of the first tubular member 110, although this is not required. In some embodiments, the second tubular member 112 may be disposed within or partially within the lumen 118 of first tubular member 110. In some instances, the second tubular member 112 may be coaxially disposed within the first tubular member 110. In other instances, the longitudinal axis of the second tubular member 112 may be offset from the first tubular member 110. In some instances, the first tubular member 110 and the second tubular member 112 may be advanced through the vasculature together. In addition, the system 100 may include one or more ablation transducers 128 positioned adjacent to the distal end region 126 of the second tubular member 112. While the ablation transducer 128 is shown and described as being positioned on the second tubular member 112, it is contemplated that in some instances, ablation transducers may be provided on the first tubular member 110. While FIGS. 2 and 3 illustrate one ablation transducer 128, it is contemplated that the modulation system 100 may include any number of ablation transducers desired, such as, but not limited to, one, two, three, or more.

In some embodiments, the ablation transducer 128 may have a cylindrical shape, however, those skilled in the art will appreciate that any suitable shapes such as, but not limited to, square, rectangular, polygonal, circular, oblong, or the like may also be contemplated. In some instances, such as when a cylindrical transducer is provided, the ablation transducer 128 may extend around the entire circumference of the second tubular member 112. In an alternative embodiment, however, the ablation transducer 128 may not extend around the entire circumference of the second tubular member 112. For instance, the ablation transducer 128 may include an array of one or more transducers (not shown) positioned about the circumference of the second tubular member 112. In other embodiments, the ablation transducer 128 may comprise a focused or phased array of transducers. The array may be configured to be directed at a focus region such that multiple transducers are radiating energy at a common target region. It is further contemplated that the ablation transducer 128 may comprise a plurality of longitudinally spaced transducers. Those skilled in the art will appreciate that other suitable configurations of the ablation transducer 128 may also be contemplated without departing from the scope and spirit of the present disclosure.

While the ablation transducer 128 is described as an ultrasonic transducer, it is contemplated that other methods and devices for raising the temperature of the nerves may be used, such as, but not limited to: radiofrequency, microwave, or other acoustic, optical, electrical current, direct contact heating, or other heating. The ablation transducer 128 may be formed from any suitable material such as, but not limited to, lead zirconate titanate (PZT). It is contemplated that other ceramic or piezoelectric materials may also be used. In some instances, the ablation transducer 128 may include a layer of gold, or other conductive layer, disposed on at least one side over the PZT crystal for connecting electrical leads to the ablation transducer 128. In some instances, one or more tie layers may be used to bond the gold to the PZT. For example, a layer of chrome may be disposed between the PZT and the gold to improve adhesion. In other instances, the transducer 128 may include a layer of chrome over the PZT followed by a layer of nickel, and finally a layer of gold. These are just examples. It is contemplated that the layers may be deposited on the PZT using sputter coating, although other deposition techniques may be used as desired.

The ablation transducer 128 may have a radiating surface, and a perimeter surface extending around the outer edge of the ablation transducer 128. The acoustic energy from the radiating surface of the ablation transducer 128 may be transmitted in a spatial pressure distribution related to the shape of the ablation transducer 128. For instance, the cylindrical shape of the ablation transducer 128 may provide a circumferential ablation pattern. In such an instance, the ablation transducer 128 may include a backing layer to direct the acoustic energy in a single direction. In other embodiments, the ablation transducer 128 may be structured to radiate acoustic energy from two radiating surfaces.

The ablation transducer 128 may be connected to a control unit (such as control unit 18 in FIG. 1) by electrical conductor(s) 140. In some embodiments, the electrical conductor(s) 140 may be disposed within a lumen of the elongated shaft 106. In other embodiments, the electrical conductor(s) 140 may extend along an outside surface of the elongated shaft 106. The electrical conductor(s) 140 may provide electricity to the ablation transducer 128, which may then be converted into acoustic energy. The acoustic energy may be directed from the ablation transducer 128 in a direction generally perpendicular to the radiating surfaces of the transducer 128. As discussed above, acoustic energy radiates from the ablation transducer 128 in a pattern related to the shape of the transducer 128 and lesions formed during ablation take shape similar to contours of the pressure distribution.

Further, the system 100 may include one or more infusion sheaths 130 having a proximal end 132, a distal end 134 and a lumen 136 extending therethrough. In some embodiments, the proximal end 132 of the infusion sheath 130 may be secured to the catheter shaft 106 adjacent to the distal end 114 of the first tubular member 110. It is contemplated that the infusion sheath 130 may be attached either temporarily or permanently to the catheter shaft 106. Suitable attachment means may include adhesives, heat shrinking, or other suitable means known to those skilled in the art. The distal end 134 of the infusion sheath 130 may be open to allow an infusion fluid 138 to exit the sheath 130. The infusion sheath 130 may be configured to extend distally from the distal end 114 of the first tubular member 110 such that a portion of the distal end region 126 of the second tubular member 112 is disposed within or partially within the lumen 136 of the infusion sheath 130. In some instances, the distal end 122 of the second tubular member 112 may extend beyond the distal end 134 of the infusion sheath 130, but this is not required. In some instances, the ablation transducer 128 may be disposed within or partially within the lumen 136 of the infusion sheath 130, although this is not required. In some instances, the lumen 136 of the infusion sheath may be in fluid communication with the lumen 118 of the first tubular member 110 for receiving an infusion fluid. Saline or other suitable infusion fluid 138 may be flushed through the infusion lumen 118 and into the lumen 136 of the infusion sheath 130. The infusion fluid 138 may displace blood from around the transducer 128. As the infusion fluid 138 flows past the ablation transducer 128, the infusion fluid 138 may provide convective cooling to the transducer 128. It is further contemplated that by displacing and/or cooling the blood surrounding the transducer 128, blood damage, fouling of the transducer 128, and/or overheating of the transducer 128 may be reduced or eliminated. In some instances, this may allow the modulation system 100 to be operated at a higher power level, thus providing a shorter treatment and/or more effective modulation of the target tissue. It is contemplated that the infusion fluid 138 may be introduced into the modulation system 100 before, during, or after ablation. Flow of the infusion fluid 138 may begin before energy is supplied to the ablation transducer 128 and continue for the duration of the modulation procedure. In some embodiments, infusion fluid may also be introduced through lumen 124 such that both the inner and outer surfaces of the ablation transducer 128 are cooled.

While not explicitly shown, in some embodiments, the infusion sheath 130 may be expanded such that it contacts the vessel wall 104. This may allow the infusion sheath 130 to provide additional cooling to the vessel wall 104 when an infusion fluid 138 is provided, which may help prevent vessel heat damage. It is contemplated that the infusion sheath 130 may be configured to be non-occluding such that it allows blood to flow past during systole (during pulse), but contacts the vessel wall 104 when there is no pulse. The flow of blood past the vessel wall 104 may provide additional cooling.

It is contemplated that the infusion sheath 130 may be formed from a material that is sonically translucent such that the ultrasound energy may pass through the infusion sheath 130. In some instances, the infusion sheath may be formed from a polymeric material having a low loss proper acoustic impedance. It is contemplated that the infusion sheath 130 may have a thickness such that significant attenuation of the ultrasound energy is avoided.

The infusion fluid 138 may be saline or any other suitable infusion fluid. It is contemplated that the infusion fluid 138 may be provided at a variety of different temperatures depending on the desired treatment. In some instances, the infusion fluid 138 may be provided at room temperature, below room temperature, above room temperature, or at normal body temperature as desired. In some instances, such as when an imaging transducer is provided (not explicitly shown), a small amount of an imaging contrast material may be added to the infusion fluid 138 to facilitate imaging of the vessel. Suitable examples of such imaging contrast material may include, but are not limited to fluorine, iodine, barium, or the like.

In some embodiments, the infusion sheath 130 may be configured to transition between an expanded state and a collapsed state. It is contemplated that the infusion sheath 130 may be self-expanding or may be expanded using an actuation mechanism, as will be discussed in more detail below with respect to FIGS. 4 and 5. In some instances, the modulation system 100 may be advanced to the treatment region within a guide catheter, such as guide catheter 16 shown in FIG. 1. Once the modulation system 100 is adjacent to the desired treatment region, the guide catheter may be retracted proximally to allow the infusion sheath 130 to expand. In some instances, the infusion fluid 138 may be provided at a flow rate and/or pressure suitable to expand the infusion sheath 130 to allow the infusion fluid 138 to exit the open distal end 134 of the infusion sheath 130. In other instances, the infusion sheath 130 may be provided with a self-expanding mechanism, such as, but not limited to, an expandable hoop or other structure positioned about the circumference of the infusion sheath 130. In some embodiments, the shape of the infusion sheath 130 may be curved, domed, umbrella, cylindrical, or the like. It may be contemplated, however, that the shape of the infusion sheath 130 may include, but is not limited to, rectangular, triangular, or the like, without limiting the scope and spirit of the present disclosure. In some instances, the diameter of the infusion sheath 130 at the distal end 134 may be larger than the diameter at the proximal end 132. It is contemplated that the diameter of the infusion sheath 130 may be varied in any number of ways, such as, but not limited to a taper or step-wise transition.

In an alternative embodiment, an infusion port (not shown) may be used in place of or in addition to the infusion sheath 130. The infusion port may be located near the proximal end of the ablation transducer 128. It is contemplated that multiple infusion holes or an annular infusion port may be provided near the proximal end of the ablation transducer 128 such that infusion fluid is directed past the ablation transducer 128. This may avoid or reduce interference that may be caused by the infusion sheath 130. In other embodiments, the distal end 134 of the infusion sheath 130 may terminate proximal of the proximal end of the ablation transducer 128. This may avoid or reduce interference that may be caused by the infusion sheath 130.

The modulation system 100 may be advanced through the vasculature in any manner known in the art. For example, system 100 may include a guidewire lumen to allow the system 100 to be advanced over a previously located guidewire. In some embodiments, the modulation system 100 may be advanced, or partially advanced, within a guide catheter such as the catheter 16 shown in FIG. 1. Once the transducer 128 of the modulation system 100 has been placed adjacent to the desired treatment area, positioning mechanisms may be deployed, if so provided. The transducer 128 may be connected to a control unit (such as control unit 18 in FIG. 1) by an electrical conductor 140. The transducer 128 may be connected to one or more control units, which may provide and/or monitor the system 100 with one or more parameters such as, but not limited to, frequency for performing the desired ablation procedure as well as imaging. In some embodiments, the electrical conductor 140 may be disposed within a lumen of the elongate shaft 106. In other embodiments, the electrical conductor 140 may be extended along an outside surface of the elongate shaft 106.

Once the modulation system 100 has been advanced to the treatment region, an infusion fluid 138 may be provided through the infusion lumen 118 and into the infusion sheath 130. It is contemplated that energy may be supplied to the ablation transducer 128 before, during, and/or after the infusion fluid 138 is provided. The electrical conductor 140 may provide electricity to the ablation transducer 128, and that energy may then be converted into acoustic energy. The acoustic energy may be directed from the ablation transducer 128 in a direction generally perpendicular to the radiating surfaces of the ablation transducer 128, generally in a pattern related to the shape of the ablation transducer 128. Although FIG. 3 illustrates a single electrical conductor 140, it is contemplated that the modulation system 100 may include any number of electrical conductors desired, such as, but not limited to, one, two, three, or more. For example, if multiple ablation transducers are provided, multiple electrical conductors may be required. The amount of energy delivered to the transducer 128 may be determined by the desired treatment as well as the feedback provided by monitoring systems.

In some instances, the elongate shaft 106 may be rotated and additional ablation can be performed at multiple locations around the circumference of the lumen 102. In some instances, a slow automated “rotisserie” rotation can be used to work around the circumference of the lumen 102, or a faster spinning can be used to simultaneously ablate around the entire circumference. The spinning can be accomplished with a distal micro-motor or by spinning a drive shaft from the proximal end. In other instances, the elongate shaft 106 may be indexed incrementally between desired orientations. In some embodiments, ultrasound sensor information can be used to selectively turn on and off the ablation transducers to warm any cool spots or accommodate for veins, or other tissue variations. The number of times the elongate shaft 106 is rotated at a given longitudinal location may be determined by the number, size and/or shape of the transducer 128 on the elongate shaft 106. Once a particular location has been ablated, it may be desirable to perform further ablation procedures at different longitudinal locations. Once the elongate shaft 106 has been longitudinally repositioned, energy may once again be delivered to the transducer 128 to perform ablation and/or imaging as desired. If necessary, the elongate shaft 106 may be rotated to perform ablation around the circumference of the lumen 102 at each longitudinal location. This process may be repeated at any number of longitudinal locations desired. It is contemplated that in some embodiments, the system 100 may include a transducer 128 at various positions along the length of the modulation system 100 such that a larger region may be treated without longitudinal displacement of the elongate shaft 106.

FIG. 4 is a schematic view of a distal end of another illustrative intravascular nerve modulation system 200 disposed within a vessel 202 having a vessel wall 204 that may be similar in form and function to other systems disclosed herein. As shown, the modulation system 200 may include a catheter shaft 206 having a distal end region 208. The catheter shaft 206 may extend proximally to a point configured to remain outside of a patient's body. The proximal end of the catheter shaft 206 may include a hub attached thereto for connecting other treatment devices or providing a port for facilitating other treatments. It is contemplated that the stiffness of the catheter shaft 206 may be modified to form a modulation system 200 for use in various vessel diameters and various locations within the vascular tree. The catheter shaft 206 may include a first tubular member 210 and a second tubular member 212. The first tubular member 210 may have a proximal end (not explicitly shown), a distal end region 214 and a lumen (not explicitly shown) extending between the proximal end and the distal end. In some embodiments, the lumen may be an infusion lumen and may be in fluid communication with an infusion fluid source configured to remain outside of a patient's body. The second tubular member 212 may have a proximal end (not shown), a distal end 216, and a lumen (not explicitly shown) extending therebetween. In some instances, the first tubular member 210 and the second tubular member 212 may be advanced through the vasculature together.

In addition, the catheter shaft 206 may have a cross-sectional configuration adapted to be received in a desired vessel, such as a renal artery. For instance, the catheter shaft 206 may specially be sized and configured to accommodate passage through the intravascular path, which leads from a percutaneous access site in, for example, the femoral, brachial, or radial artery, to a targeted treatment site, for example, within a renal artery. An exemplary embodiment may depict the catheter shaft 206 to take on a long, thin, flexible tube-shaped structure having a tubular cross-section; however, other contemplated cross-sections may include rectangular, irregular, or other suitable structures known to those skilled in the art.

The catheter shaft 206 may further include one or more lumens (not explicitly shown). For example, the catheter shaft 206 may include a guidewire lumen and/or one or more auxiliary lumens. The lumens may be configured in any suitable way such as those ways commonly used for medical device. For example, the guidewire lumen may extend the entire length of the catheter shaft 206 such as in an over-the-wire catheter or may extend only along a distal portion of the catheter shaft 206 such as in a single operator exchange (SOE) catheter. These examples are not intended to be limiting, but rather examples of some possible configurations. While not explicitly shown, the modulation system 200 may further include temperature sensor/wire, an infusion lumen, radiopaque marker bands, fixed guidewire tip, a guidewire lumen, external sheath, and/or other components to facilitate the use and advancement of the system 200 within the vasculature.

The modulation system 200 may further include one or more ablation transducers 218 disposed adjacent the distal end region 220 of the second tubular member 212. While the ablation transducer 218 is shown and described as being positioned on the second tubular member 212, it is contemplated that in some instances, ablation transducers may be provided on the first tubular member 210. The ablation transducer 218 may be formed from any suitable material such as, but not limited to, lead zirconate titanate (PZT). It is contemplated that other ceramic or piezoelectric materials may also be used. It is contemplated that the transducer 218 may have similar form and function to the transducer 128 discussed above. In some embodiments, the ablation transducer 218 may have a cylindrical shape and extend around the entire circumference of the second tubular member 212. In other embodiments, there may be any number of ablation transducers 218 (one, two, three, four, or more) spaced about the circumference of the second tubular member 212. This may allow for ablation of multiple radial locations about the body lumen simultaneously. In other embodiments, the ablation transducer 218 may comprise a focused or phased array of transducers. The array may be configured to be directed at a focus region such that multiple transducers are radiating energy at a common target region. It is further contemplated that the ablation transducer 218 may comprise a plurality of longitudinally spaced transducers.

The ablation transducer 218 may be connected to a control unit (such as control unit 18 in FIG. 1) by electrical conductor(s). In some embodiments, the electrical conductor(s) may be disposed within a lumen of the elongated shaft 206. In other embodiments, the electrical conductor(s) may extend along an outside surface of the elongated shaft 206. The electrical conductor(s) may provide electricity to the ablation transducer 218, which may then be converted into acoustic energy. The acoustic energy may be directed from the ablation transducer 218 in a direction generally perpendicular to the radiating surfaces of the transducer 218. As discussed above, acoustic energy radiates from the ablation transducer 218 in a pattern related to the shape of the transducer 218 and lesions formed during ablation take shape similar to contours of the pressure distribution.

Further, the system 200 may include one or more infusion sheaths 222 having a proximal end 224, a distal end 226 and a lumen 228 extending therethrough. The infusion sheath 222 may have similar form and function to the infusion sheath 130 discussed above. In some embodiments, the proximal end 224 of the infusion sheath 222 may be secured to the catheter shaft 206 adjacent to the distal end region 214 of the first tubular member 210. It is contemplated that the infusion sheath 222 may be attached either temporarily or permanently to the catheter shaft 206. The distal end 226 of the infusion sheath 222 may be open to allow an infusion fluid to exit the sheath 222. The infusion sheath 222 may be configured to extend distally from the distal end region 214 of the first tubular member 210 such that a portion of the distal end region 220 of the second tubular member 212 is disposed within or partially within the lumen 228 of the infusion sheath 222. In some instances, the distal end 216 of the second tubular member 212 may extend beyond the distal end 226 of the infusion sheath 222, but this is not required. In some instances, the ablation transducer 218 may be disposed within or partially within the lumen 228 of the infusion sheath 222, although this is not required. In some instances, the lumen 228 of the infusion sheath may be in fluid communication with a lumen of the first tubular member 210 for receiving an infusion fluid. Saline or other suitable infusion fluid may be flushed through an infusion lumen of the elongate shaft 206 and into the lumen 228 of the infusion sheath. The infusion fluid may displace blood from around the transducer 218. As the infusion fluid flows past the ablation transducer 218, the infusion fluid may provide convective cooling to the transducer 218. It is further contemplated that by displacing and/or cooling the blood surrounding the transducer 218, blood damage, fouling of the transducer 218, and/or overheating of the transducer 218 may be reduced or eliminated. In some instances, this may allow the modulation system 200 to be operated at a higher power level, thus providing a shorter treatment and/or more effective modulation of the target tissue. It is contemplated that the infusion fluid may be introduced into the modulation system 200 before, during, or after ablation. Flow of the infusion fluid may begin before energy is supplied to the ablation transducer 218 and continue for the duration of the modulation procedure.

It is contemplated that the infusion sheath 222 may be formed from a material that is sonically translucent such that the ultrasound energy may pass through the infusion sheath 222. In some instances, the infusion sheath may be formed from a polymeric material having a low loss proper acoustic impedance. It is contemplated that the infusion sheath 222 may have a thickness such that significant attenuation of the ultrasound energy is avoided.

In some embodiments, the infusion sheath 222 may be configured to transition between an expanded state and a collapsed state. It is contemplated that the infusion sheath 222 may be self-expanding or may be expanded using an actuation mechanism. The infusion sheath 222 may include one or more longitudinally extending reinforcing filaments 230 configured to provide reinforcement to the sheath 222 while still allowing it to collapse. It is contemplated that the infusion sheath may be provided with any number of reinforcing filaments 230 desired, such as, but not limited to, one, two, three, four, or more. The reinforcing filaments 230 may be formed from any material desired, such as, but not limited to, polymers, metals, metal alloys, shape memory materials, etc. In some instances, the reinforcing filaments 230 may be formed with the infusion sheath 222. For example, the infusion sheath 222 may be extruded with longitudinal lines having a thicker profile than the remaining portions of the sheath 222.

In some instances, the modulation system 200 may be advanced to the treatment region within a guide catheter, such as guide catheter 16 shown in FIG. 1. Once the modulation system 200 is adjacent to the desired treatment region, the guide catheter may be retracted proximally to allow the infusion sheath 222 to expand. In some instances, the infusion fluid may be provided at a flow rate and/or pressure suitable to expand the infusion sheath 222 to allow the infusion fluid to exit the open distal end 226 of the infusion sheath 222. In other instances, the reinforcing filaments 230 may impart a self-expanding or self-collapsing tendency. For example, it is contemplated that the reinforcing filaments 230 may be formed from a shape memory material such as nitinol, which may bias the infusion sheath 222 into an expanded or collapsed configuration. In some embodiments, the reinforcing filaments 230 may be attached to a pull wire or other actuating member to allow a push-pull actuation force to expand or collapse the infusion sheath 222. Allowing a user to control when the infusion sheath 222 is expanded or collapsed may allow the modulation system 200 to be advanced through the vasculature without the use of a guide catheter or other introduction or removal sheath.

FIG. 5 is a schematic view of a distal end of another illustrative intravascular nerve modulation system 300 disposed within a vessel 302 having a vessel wall 304 that may be similar in form and function to other systems disclosed herein. As shown, the modulation system 300 may include a catheter shaft 306 having a distal end region 308. The catheter shaft 306 may extend proximally to a point configured to remain outside of a patient's body. The proximal end of the catheter shaft 306 may include a hub attached thereto for connecting other treatment devices or providing a port for facilitating other treatments. It is contemplated that the stiffness of the catheter shaft 306 may be modified to form a modulation system 300 for use in various vessel diameters and various locations within the vascular tree. The catheter shaft 306 may include a first tubular member 310 and a second tubular member 312. The first tubular member 310 may have a proximal end (not explicitly shown), a distal end region 314 and a lumen (not explicitly shown) extending between the proximal end and the distal end. In some embodiments, the lumen may be an infusion lumen and may be in fluid communication with an infusion fluid source configured to remain outside of a patient's body. The second tubular member 312 may have a proximal end (not shown), a distal end 316, and a lumen (not explicitly shown) extending therebetween. In some instances, the first tubular member 310 and the second tubular member 312 may be advanced through the vasculature together.

In addition, the catheter shaft 306 may have a cross-sectional configuration adapted to be received in a desired vessel, such as a renal artery. For instance, the catheter shaft 306 may specially be sized and configured to accommodate passage through the intravascular path, which leads from a percutaneous access site in, for example, the femoral, brachial, or radial artery, to a targeted treatment site, for example, within a renal artery. An exemplary embodiment may depict the catheter shaft 306 to take on a long, thin, flexible tube-shaped structure having a tubular cross-section; however, other contemplated cross-sections may include rectangular, irregular, or other suitable structures known to those skilled in the art.

The catheter shaft 306 may further include one or more lumens (not explicitly shown). For example, the catheter shaft 306 may include a guidewire lumen and/or one or more auxiliary lumens. The lumens may be configured in any suitable way such as those ways commonly used for medical device. For example, the guidewire lumen may extend the entire length of the catheter shaft 306 such as in an over-the-wire catheter or may extend only along a distal portion of the catheter shaft 306 such as in a single operator exchange (SOE) catheter. These examples are not intended to be limiting, but rather examples of some possible configurations. While not explicitly shown, the modulation system 300 may further include temperature sensor/wire, an infusion lumen, radiopaque marker bands, fixed guidewire tip, a guidewire lumen, external sheath, and/or other components to facilitate the use and advancement of the system 300 within the vasculature.

The modulation system 300 may further include one or more ablation transducers 318 disposed adjacent the distal end region 320 of the second tubular member 312. While the ablation transducer 318 is shown and described as being positioned on the second tubular member 312, it is contemplated that in some instances, ablation transducers may be provided on the first tubular member 310. The ablation transducer 318 may be formed from any suitable material such as, but not limited to, lead zirconate titanate (PZT). It is contemplated that other ceramic or piezoelectric materials may also be used. It is contemplated that the transducer 318 may have similar form and function to the transducer 128 discussed above. In some embodiments, the ablation transducer 318 may have a cylindrical shape and extend around the entire circumference of the second tubular member 312. In other embodiments, there may be any number of ablation transducers 318 (one, two, three, four, or more) spaced about the circumference of the second tubular member 312. This may allow for ablation of multiple radial locations about the body lumen simultaneously. In other embodiments, the ablation transducer 318 may comprise a focused or phased array of transducers. The array may be configured to be directed at a focus region such that multiple transducers are radiating energy at a common target region. It is further contemplated that the ablation transducer 318 may comprise a plurality of longitudinally spaced transducers.

The ablation transducer 318 may be connected to a control unit (such as control unit 18 in FIG. 1) by electrical conductor(s). In some embodiments, the electrical conductor(s) may be disposed within a lumen of the elongated shaft 306. In other embodiments, the electrical conductor(s) may extend along an outside surface of the elongated shaft 306. The electrical conductor(s) may provide electricity to the ablation transducer 318, which may then be converted into acoustic energy. The acoustic energy may be directed from the ablation transducer 318 in a direction generally perpendicular to the radiating surfaces of the transducer 318. As discussed above, acoustic energy radiates from the ablation transducer 318 in a pattern related to the shape of the transducer 318 and lesions formed during ablation take shape similar to contours of the pressure distribution.

Further, the system 300 may include one or more infusion sheaths 322 having a proximal end 324, a distal end 326 and a lumen 328 extending therethrough. The infusion sheath 322 may have similar form and function to the infusion sheath 130 discussed above. In some embodiments, the proximal end 324 of the infusion sheath 322 may be secured to the catheter shaft 306 adjacent to the distal end region 314 of the first tubular member 310. It is contemplated that the infusion sheath 322 may be attached either temporarily or permanently to the catheter shaft 306. The distal end 326 of the infusion sheath 322 may be open to allow an infusion fluid to exit the sheath 322. The infusion sheath 322 may be configured to extend distally from the distal end region 314 of the first tubular member 310 such that a portion of the distal end region 320 of the second tubular member 312 is disposed within or partially within the lumen 328 of the infusion sheath 322. In some instances, the distal end 316 of the second tubular member 312 may extend beyond the distal end 326 of the infusion sheath 322, but this is not required. In some instances, the ablation transducer 318 may be disposed within or partially within the lumen 328 of the infusion sheath 322, although this is not required. In some instances, the lumen 328 of the infusion sheath may be in fluid communication with a lumen of the first tubular member 310 for receiving an infusion fluid. Saline or other suitable infusion fluid may be flushed through an infusion lumen of the elongate shaft 306 and into the lumen 328 of the infusion sheath. The infusion fluid may displace blood from around the transducer 318. As the infusion fluid flows past the ablation transducer 318, the infusion fluid may provide convective cooling to the transducer 318. It is further contemplated that by displacing and/or cooling the blood surrounding the transducer 318, blood damage, fouling of the transducer 318, and/or overheating of the transducer 318 may be reduced or eliminated. In some instances, this may allow the modulation system 300 to be operated at a higher power level, thus providing a shorter treatment and/or more effective modulation of the target tissue. It is contemplated that the infusion fluid may be introduced into the modulation system 300 before, during, or after ablation. Flow of the infusion fluid may begin before energy is supplied to the ablation transducer 318 and continue for the duration of the modulation procedure.

It is contemplated that the infusion sheath 322 may be formed from a material that is sonically translucent such that the ultrasound energy may pass through the infusion sheath 322. In some instances, the infusion sheath may be formed from a polymeric material having a low loss proper acoustic impedance. It is contemplated that the infusion sheath 322 may have a thickness such that significant attenuation of the ultrasound energy is avoided.

In some embodiments, the infusion sheath 322 may be configured to transition between an expanded state and a collapsed state. It is contemplated that the infusion sheath 322 may be self-expanding or may be expanded using an actuation mechanism. The infusion sheath 322 may include one or more helically wound reinforcing filaments 330 configured to provide reinforcement to the sheath 322 while still allowing it to collapse. Various configurations of reinforcing filaments 330 may be selected based on the desired application. For example, the reinforcing filaments 330 may be braided, have a shape similar to a stent, extend longitudinally, extend longitudinally with some circumferential zig-zags, etc. These are only examples; the reinforcing filaments 330 may have any configuration desired. It is contemplated that the infusion sheath may be provided with any number of reinforcing filaments 330 desired, such as, but not limited to, one, two, three, four, or more. The reinforcing filaments may be formed from any material desired, such as, but not limited to, polymers, metals, metal alloys, shape memory materials, etc.

In some instances, the modulation system 300 may be advanced to the treatment region within a guide catheter, such as guide catheter 16 shown in FIG. 1. Once the modulation system 300 is adjacent to the desired treatment region, the guide catheter may be retracted proximally to allow the infusion sheath 322 to expand. In some instances, the infusion fluid may be provided at a flow rate and/or pressure suitable to expand the infusion sheath 322 to allow the infusion fluid to exit the open distal end 326 of the infusion sheath 322. In other instances, the reinforcing filaments 330 may impart a self-expanding or self-collapsing tendency. For example, it is contemplated that the reinforcing filaments 330 may be formed from a shape memory material such as nitinol, which may bias the infusion sheath 322 into an expanded or collapsed configuration. In some embodiments, the reinforcing filaments 330 may be attached to a pull wire or other actuating member to allow a push-pull actuation force to expand or collapse the infusion sheath 322. Allowing a user to control when the infusion sheath 322 is expanded or collapsed may allow the modulation system 300 to be advanced through the vasculature without the use of a guide catheter or other introduction or removal sheath.

FIG. 6 is a schematic view of a distal end of another illustrative intravascular nerve modulation system 400 disposed within a vessel 402 having a vessel wall 404 that may be similar in form and function to other systems disclosed herein. As shown, the modulation system 400 may include a catheter shaft 406 having a distal end region 408. The catheter shaft 406 may extend proximally to a point configured to remain outside of a patient's body. The proximal end of the catheter shaft 406 may include a hub attached thereto for connecting other treatment devices or providing a port for facilitating other treatments. It is contemplated that the stiffness of the catheter shaft 406 may be modified to form a modulation system 400 for use in various vessel diameters and various locations within the vascular tree. The catheter shaft 406 may include a first tubular member 410 and a second tubular member 412. The first tubular member 410 may have a proximal end (not explicitly shown), a distal end region 414 and a lumen (not explicitly shown) extending between the proximal end and the distal end. In some embodiments, the lumen may be an infusion lumen and may be in fluid communication with an infusion fluid source configured to remain outside of a patient's body. The second tubular member 412 may have a proximal end (not shown), a distal end 416, and a lumen (not explicitly shown) extending therebetween. In some instances, the first tubular member 410 and the second tubular member 412 may be advanced through the vasculature together.

In addition, the catheter shaft 406 may have a cross-sectional configuration adapted to be received in a desired vessel, such as a renal artery. For instance, the catheter shaft 406 may specially be sized and configured to accommodate passage through the intravascular path, which leads from a percutaneous access site in, for example, the femoral, brachial, or radial artery, to a targeted treatment site, for example, within a renal artery. An exemplary embodiment may depict the catheter shaft 406 to take on a long, thin, flexible tube-shaped structure having a tubular cross-section; however, other contemplated cross-sections may include rectangular, irregular, or other suitable structures known to those skilled in the art.

The catheter shaft 406 may further include one or more lumens (not explicitly shown). For example, the catheter shaft 406 may include a guidewire lumen and/or one or more auxiliary lumens. The lumens may be configured in any suitable way such as those ways commonly used for medical device. For example, the guidewire lumen may extend the entire length of the catheter shaft 406 such as in an over-the-wire catheter or may extend only along a distal portion of the catheter shaft 406 such as in a single operator exchange (SOE) catheter. These examples are not intended to be limiting, but rather examples of some possible configurations. While not explicitly shown, the modulation system 400 may further include temperature sensor/wire, an infusion lumen, radiopaque marker bands, fixed guidewire tip, a guidewire lumen, external sheath, and/or other components to facilitate the use and advancement of the system 400 within the vasculature.

The modulation system 400 may further include one or more ablation transducers 418 disposed adjacent the distal end region 420 of the second tubular member 412. While the ablation transducer 418 is shown and described as being positioned on the second tubular member 412, it is contemplated that in some instances, ablation transducers may be provided on the first tubular member 410. The ablation transducer 418 may be formed from any suitable material such as, but not limited to, lead zirconate titanate (PZT). It is contemplated that other ceramic or piezoelectric materials may also be used. It is contemplated that the transducer 418 may have similar form and function to the transducer 128 discussed above. In some embodiments, the ablation transducer 418 may have a cylindrical shape and extend around the entire circumference of the second tubular member 412. In other embodiments, there may be any number of ablation transducers 418 (one, two, three, four, or more) spaced about the circumference of the second tubular member 412. This may allow for ablation of multiple radial locations about the body lumen simultaneously. In other embodiments, the ablation transducer 418 may comprise a focused or phased array of transducers. The array may be configured to be directed at a focus region such that multiple transducers are radiating energy at a common target region. It is further contemplated that the ablation transducer 418 may comprise a plurality of longitudinally spaced transducers.

The ablation transducer 418 may be connected to a control unit (such as control unit 18 in FIG. 1) by electrical conductor(s). In some embodiments, the electrical conductor(s) may be disposed within a lumen of the elongated shaft 406. In other embodiments, the electrical conductor(s) may extend along an outside surface of the elongated shaft 406. The electrical conductor(s) may provide electricity to the ablation transducer 418, which may then be converted into acoustic energy. The acoustic energy may be directed from the ablation transducer 418 in a direction generally perpendicular to the radiating surfaces of the transducer 418. As discussed above, acoustic energy radiates from the ablation transducer 418 in a pattern related to the shape of the transducer 418 and lesions formed during ablation take shape similar to contours of the pressure distribution.

Further, the system 400 may include one or more infusion sheaths 422 having a proximal end 424, a distal end 426 and a lumen 428 extending therethrough. The infusion sheath 422 may have similar form and function to the infusion sheath 130 discussed above. In some embodiments, the proximal end 424 of the infusion sheath 422 may be secured to the catheter shaft 406 adjacent to the distal end region 414 of the first tubular member 410. It is contemplated that the infusion sheath 422 may be attached either temporarily or permanently to the catheter shaft 406. The distal end 426 of the infusion sheath 422 may be open to allow an infusion fluid to exit the sheath 422. The infusion sheath 422 may be configured to extend distally from the distal end region 414 of the first tubular member 410 such that a portion of the distal end region 420 of the second tubular member 412 is disposed within or partially within the lumen 428 of the infusion sheath 422. In some instances, the distal end 416 of the second tubular member 412 may extend beyond the distal end 426 of the infusion sheath 422, but this is not required. In some instances, the ablation transducer 418 may be disposed within or partially within the lumen of the infusion sheath 422, although this is not required.

In some instances, the lumen 428 of the infusion sheath may be in fluid communication with a lumen of the first tubular member 410 for receiving an infusion fluid 432. Saline or other suitable infusion fluid may be flushed through an infusion lumen of the elongate shaft 406 and into the lumen 428 of the infusion sheath. The infusion fluid 432 may displace blood from around the transducer 418. As the infusion fluid 432 flows past the ablation transducer 418, the infusion fluid 432 may provide convective cooling to the transducer 418. In some embodiments, the infusion sheath 422 may be provided with side holes or apertures 430 to direct the infusion fluid outward toward the vessel wall 404. The side holes 430 may be sized and shaped to allow infusion fluid 432 to exit the infusion sheath 422 proximal to the distal end 426 opening. This may provide additional cooling of the vessel wall 404 to protect the wall 404 from injure due to conduction of heat from the deeper target tissue. For clarity, not all of the side holes 430 have been identified with a reference number in FIG. 6. It is contemplated that the infusion sheath 422 may be provided with any number of side holes 430 desired. Additionally, the side holes 430 may be provided in any pattern, uniform or non-uniform, desired.

It is further contemplated that by displacing and/or cooling the blood surrounding the transducer 418, blood damage, fouling of the transducer 418, and/or overheating of the transducer 418 may be reduced or eliminated. In some instances, this may allow the modulation system 400 to be operated at a higher power level, thus providing a shorter treatment and/or more effective modulation of the target tissue. It is contemplated that the infusion fluid may be introduced into the modulation system 400 before, during, or after ablation. Flow of the infusion fluid may begin before energy is supplied to the ablation transducer 418 and continue for the duration of the modulation procedure.

It is contemplated that the infusion sheath 422 may be formed from a material that is sonically translucent such that the ultrasound energy may pass through the infusion sheath 422. In some instances, the infusion sheath may be formed from a polymeric material having a low loss proper acoustic impedance. It is contemplated that the infusion sheath 422 may have a thickness such that significant attenuation of the ultrasound energy is avoided.

In some embodiments, the infusion sheath 422 may be configured to transition between an expanded state and a collapsed state. It is contemplated that the infusion sheath 422 may be self-expanding or may be expanded using an actuation mechanism as discussed above. In some instances, the modulation system 400 may be advanced to the treatment region within a guide catheter, such as guide catheter 16 shown in FIG. 1. Once the modulation system 400 is adjacent to the desired treatment region, the guide catheter may be retracted proximally to allow the infusion sheath 422 to expand. In some instances, the infusion fluid may be provided at a flow rate and/or pressure suitable to expand the infusion sheath 422 to allow the infusion fluid to exit the open distal end 426 of the infusion sheath 422. FIG. 6 is a schematic view of a distal end of another illustrative intravascular nerve modulation system 500 disposed within a vessel 502 having a vessel wall 504 that may be similar in form and function to other systems disclosed herein. As shown, the modulation system 500 may include a catheter shaft 506 having a distal end region 508. The catheter shaft 506 may extend proximally to a point configured to remain outside of a patient's body. The proximal end of the catheter shaft 506 may include a hub attached thereto for connecting other treatment devices or providing a port for facilitating other treatments. It is contemplated that the stiffness of the catheter shaft 506 may be modified to form a modulation system 500 for use in various vessel diameters and various locations within the vascular tree. The catheter shaft 506 may include a first tubular member 510 and a second tubular member 512. The first tubular member 510 may have a proximal end (not explicitly shown), a distal end region 514 and a lumen 532 extending between the proximal end and the distal end. In some embodiments, the lumen may be an infusion lumen and may be in fluid communication with an infusion fluid source configured to remain outside of a patient's body. The second tubular member 512 may have a proximal end (not shown), a distal end 516, and a lumen 534 extending therebetween. In some instances, the first tubular member 510 and the second tubular member 512 may be advanced through the vasculature together. The first tubular member 510 and second tubular member 512 may be positioned side-by-side configuration. In some embodiments, the first tubular member 510 and second tubular member 512 may be formed as a unitary elongate member. In other embodiments, the first and second tubular members 510, 512 may be formed as separate members and subsequently joined.

In addition, the catheter shaft 506 may have a cross-sectional configuration adapted to be received in a desired vessel, such as a renal artery. For instance, the catheter shaft 506 may specially be sized and configured to accommodate passage through the intravascular path, which leads from a percutaneous access site in, for example, the femoral, brachial, or radial artery, to a targeted treatment site, for example, within a renal artery. An exemplary embodiment may depict the catheter shaft 506 to take on a long, thin, flexible tube-shaped structure having a tubular cross-section; however, other contemplated cross-sections may include rectangular, irregular, or other suitable structures known to those skilled in the art.

The catheter shaft 506 may further include one or more lumens, such as lumens 532, 534. For example, the catheter shaft 506 may include a guidewire lumen and/or one or more auxiliary lumens. The lumens may be configured in any suitable way such as those ways commonly used for medical device. For example, the guidewire lumen may extend the entire length of the catheter shaft 506 such as in an over-the-wire catheter or may extend only along a distal portion of the catheter shaft 506 such as in a single operator exchange (SOE) catheter. These examples are not intended to be limiting, but rather examples of some possible configurations. While not explicitly shown, the modulation system 500 may further include temperature sensor/wire, an infusion lumen, radiopaque marker bands, fixed guidewire tip, a guidewire lumen, external sheath, and/or other components to facilitate the use and advancement of the system 500 within the vasculature.

The modulation system 500 may further include one or more ablation transducers 518 disposed adjacent the distal end region 520 of the second tubular member 512. While the ablation transducer 518 is shown and described as being positioned on the second tubular member 512, it is contemplated that in some instances, ablation transducers 518 may be provided on the first tubular member 510. The ablation transducer 518 may be formed from any suitable material such as, but not limited to, lead zirconate titanate (PZT). It is contemplated that other ceramic or piezoelectric materials may also be used. It is contemplated that the transducer 518 may have similar form and function to the transducer 128 discussed above. In some embodiments, the ablation transducer 518 may have a cylindrical shape and extend around the entire circumference of the second tubular member 512. In other embodiments, there may be any number of ablation transducers 518 (one, two, three, four, or more) spaced about the circumference of the second tubular member 512. This may allow for ablation of multiple radial locations about the body lumen 502 simultaneously. In other embodiments, the ablation transducer 518 may comprise a focused or phased array of transducers. The array may be configured to be directed at a focus region such that multiple transducers are radiating energy at a common target region. It is further contemplated that the ablation transducer 518 may comprise a plurality of longitudinally spaced transducers.

The ablation transducer 518 may be connected to a control unit (such as control unit 18 in FIG. 1) by electrical conductor(s). In some embodiments, the electrical conductor(s) may be disposed within a lumen of the elongated shaft 506. In other embodiments, the electrical conductor(s) may extend along an outside surface of the elongated shaft 506. The electrical conductor(s) may provide electricity to the ablation transducer 518, which may then be converted into acoustic energy. The acoustic energy may be directed from the ablation transducer 518 in a direction generally perpendicular to the radiating surfaces of the transducer 518. As discussed above, acoustic energy radiates from the ablation transducer 518 in a pattern related to the shape of the transducer 518 and lesions formed during ablation take shape similar to contours of the pressure distribution.

Further, the system 500 may include one or more infusion sheaths 522 having a proximal end 524, a distal end 526 and a lumen 528 extending therethrough. The infusion sheath 522 may have similar form and function to the infusion sheath 130 discussed above. In some embodiments, the proximal end 524 of the infusion sheath 522 may be secured to the catheter shaft 506 adjacent to the distal end region 514 of the first tubular member 510 and proximal to the distal end 516 of the second tubular member. The infusion sheath may be secured to both the first tubular member 510 and the second tubular member 512. It is contemplated that the infusion sheath 522 may be attached either temporarily or permanently to the catheter shaft 506. The distal end 526 of the infusion sheath 522 may be open to allow an infusion fluid 530 to exit the sheath 522. The infusion sheath 522 may be configured to extend distally from the distal end region 514 of the first tubular member 510 such that a portion of the distal end region 520 of the second tubular member 512 is disposed within or partially within the lumen 528 of the infusion sheath 522. In some instances, the distal end 516 of the second tubular member 512 may extend beyond the distal end 526 of the infusion sheath 522, but this is not required. In some instances, the ablation transducer 518 may be disposed within or partially within the lumen 528 of the infusion sheath 522, although this is not required. In some instances, the lumen 528 of the infusion sheath may be in fluid communication with the lumen 532 of the first tubular member 510 for receiving an infusion fluid 530. Saline or other suitable infusion fluid may be flushed through an infusion lumen 532 of the elongate shaft 506 and into the lumen 528 of the infusion sheath 522. The infusion fluid 530 may displace blood from around the transducer 518. As the infusion fluid 530 flows past the ablation transducer 518, the infusion fluid 530 may provide convective cooling to the transducer 518. It is further contemplated that by displacing and/or cooling the blood surrounding the transducer 518, blood damage, fouling of the transducer 518, and/or overheating of the transducer 518 may be reduced or eliminated. In some instances, this may allow the modulation system 500 to be operated at a higher power level, thus providing a shorter treatment and/or more effective modulation of the target tissue. It is contemplated that the infusion fluid may be introduced into the modulation system 500 before, during, or after ablation. Flow of the infusion fluid may begin before energy is supplied to the ablation transducer 518 and continue for the duration of the modulation procedure.

It is contemplated that the infusion sheath 522 may be formed from a material that is sonically translucent such that the ultrasound energy may pass through the infusion sheath 522. In some instances, the infusion sheath may be formed from a polymeric material having a low loss proper acoustic impedance. It is contemplated that the infusion sheath 522 may have a thickness such that significant attenuation of the ultrasound energy is avoided.

In some embodiments, the infusion sheath 522 may be configured to transition between an expanded state and a collapsed state. It is contemplated that the infusion sheath 522 may be self-expanding or may be expanded using an actuation mechanism as discussed above. In some instances, the modulation system 500 may be advanced to the treatment region within a guide catheter, such as guide catheter 16 shown in FIG. 1. Once the modulation system 500 is adjacent to the desired treatment region, the guide catheter may be retracted proximally to allow the infusion sheath 522 to expand. In some instances, the infusion fluid may be provided at a flow rate and/or pressure suitable to expand the infusion sheath 522 to allow the infusion fluid to exit the open distal end 526 of the infusion sheath 522.

In an alternative embodiment, an infusion port (not shown) may be used in place of or in addition to the infusion sheath 522. The infusion port may be located near the proximal end of the ablation transducer 518. It is contemplated that multiple infusion holes or an annular infusion port may be provided near the proximal end of the ablation transducer 518 such that infusion fluid is directed past the ablation transducer 518. This may avoid or reduce interference that may be caused by the infusion sheath 522. In other embodiments, the distal end 526 of the infusion sheath 522 may terminate proximal of the proximal end of the ablation transducer 518. This may avoid or reduce interference that may be caused by the infusion sheath 522.

Those skilled in the art will recognize that the present invention may be manifested in a variety of forms other than the specific embodiments described and contemplated herein. Accordingly, departure in form and detail may be made without departing from the scope and spirit of the present disclosure as described in the appended claims.

What is claimed is:

1. An intravascular nerve modulation system, comprising:
a catheter shaft including a first tubular member having a proximal end and a distal end and a second tubular member having a proximal end and a distal end, the distal end of the second tubular member extending distally beyond the distal end of the first tubular member;
at least one ablation transducer coupled to a distal end region of the second tubular member; and
an infusion sheath having a proximal end and a distal end, the proximal end of the infusion sheath secured to the catheter shaft adjacent the distal end of the first tubular member.

2. The intravascular nerve modulation system of claim 1, wherein the first tubular member defines an infusion lumen.

3. The intravascular nerve modulation system claim 1, wherein the infusion sheath is disposed over the at least one ablation transducer.

4. The intravascular nerve modulation system of claim 1, wherein the distal end of the infusion sheath is open.

5. The intravascular nerve modulation system of claim 1, wherein the infusion sheath comprises a sonically translucent material.

6. The intravascular nerve modulation system of claim 1, wherein the at least one ablation transducer comprises a cylindrical transducer.

7. The intravascular nerve modulation system of claim 1, wherein the at least one ablation transducer comprises an ultrasound transducer.

8. The intravascular nerve modulation system of claim 1, wherein the infusion sheath further comprises one or more reinforcing filaments extending along a length thereof.

9. The intravascular nerve modulation system of claim 3, wherein the infusion sheath comprises a plurality of side holes.

10. An intravascular nerve modulation system comprising:
an outer tubular member having a proximal end and a distal end and a lumen extending therebetween;
an inner tubular member disposed within the lumen of the outer tubular member, the inner tubular member having a proximal end and a distal end region, the distal end region of the inner tubular member extending distally beyond the distal end of the outer tubular member;
at least one ablation transducer affixed to the distal end region of the inner tubular member;
an infusion sheath having a proximal end and a distal end, the proximal end of the infusion sheath fixedly secured adjacent to the distal end of the outer tubular member.

11. The intravascular nerve modulation system of claim 10, wherein the infusion sheath is disposed over the at least one ablation transducer.

12. The intravascular nerve modulation system of claim 11, wherein the lumen of the outer tubular member is configured to transport an infusion fluid from the proximal end of the outer tubular member to the distal end of the outer tubular member and into the infusion sheath.

13. The intravascular nerve modulation system of claim 12, wherein the infusion fluid surrounds the at least one ablation transducer.

14. The intravascular nerve modulation system of claim 10, wherein the infusion sheath comprises a sonically translucent material.

15. The intravascular nerve modulation system of claim 10, wherein the infusion sheath further comprises one or more reinforcing filaments extending along a length thereof.

16. An intravascular nerve modulation system comprising:
a first tubular member having a proximal end and a distal end and a lumen extending therebetween;
a second tubular member extending longitudinally along the first tubular member, the second tubular member having a proximal end and a distal end region, the distal end region of the second tubular member extending distally beyond the distal end of the first tubular member;
an ultrasound transducer affixed to the distal end region of the second tubular member;
an infusion sheath having a proximal end and a distal end, the proximal end of the infusion sheath fixedly secured adjacent to the distal end of the first tubular member.

17. The intravascular nerve modulation system of claim 16, wherein the proximal end of the infusion sheath is secured to both the first tubular member and the second tubular member.

18. The intravascular nerve modulation system of claim 16, wherein the lumen of the first tubular member is configured to transport an infusion fluid from the proximal end of the first tubular member to the distal end of the first tubular member and into the infusion sheath.

19. The intravascular nerve modulation system of claim 18, wherein the infusion fluid surrounds the ultrasound transducer.

20. The intravascular nerve modulation system of claim 16, wherein the infusion sheath comprises a sonically translucent material.
